(19) United States
(12) Patent Application Publication
(10) Pub. No.: US 2008/0103486 A1
(43) Pub. Date: May 1, 2008

(54) SafeFeed Adapter

(76) Inventor: Troy Jean-Luc Owens, Chula Vista, CA (US)

Correspondence Address: Troy Jean-Luc Owens, 1029 Hawthorne Creek Drive, Chula Vista, CA, 91914, US

(21) Appl. No.: 11/975,876

(22) Filed: Oct. 22, 2007

Related U.S. Application Data
(60) Provisional application No. 60/854,531, filed on Oct. 26, 2006.

(51) Int. Cl. A61M 39/12

(52) U.S. Cl. 604/533

(57) ABSTRACT

The present invention provides a connector with a first end portion and a second end portion that contains an indentation for providing enteral feedings that may prevent tube misconnections of an enteral feeding set to an IV catheter or rigid female luer connector.

### BACKGROUND OF THE INVENTION

The present invention relates generally to the field of medical device. In particular, the present invention relates to an adapter utilized for connecting specific types of medical devices.

The best way to eliminate risk of inadvertent use of an enteral adapter “unintentionally connecting one end of a tube or catheter to the wrong tube or device” would require a manufacturing change in the distal end of the current design of the enteral tubing set. Currently, some enteral access devices are constructed to be compatible or mate with Central Intravenous Catheters, Central Venous pressure parts, Infusion ports, Balloon ports, Introducer ports, IV Luer Connectors, Peritoneal Dialysis Catheters, Distal Port for a Pulmonary Artery Catheter, IV Luer connectors, and many other fittings, and one unintended consequence of these adapters is that they provide a link between two unrelated systems, i.e., enteral to intravenous (IV). These systems are intended to have unique methods of delivery, with distinctly different purposes, which current adapters can circumvent—possibly resulting in harm or serious injury to the patient.

Joint Commission on Accreditation of Healthcare Organizations (JCAHO), a non-profit organization with a mission to maintain and elevate the standards of health care delivery through evaluation and accreditation of healther care organizations, issued a Sentinel Alert report on Apr. 3, 2006, titled, “Tubing misconnections—a persistent and potentially deadly occurrence.” To date, nine cases involving tubing misconnections have been reported to JCAHO's Sentinel Event Database. Reports in the media and to organizations such as ECRI, the Food and Drug Administration (FDA), the Institute for Safe Medication Practices (ISMP), and United States Pharmacopeia (USP) indicate that misconnection errors occur with significant frequency and, in a number of instances, lead to fatal consequences.

JCAHO's Sentinel Event Alert, issue 36, provided their first recommendation and strategy to healthcare organizations to reduce tubing misconnection errors by suggesting not to purchase types of non-intravenous equipment that contains connectors that can mate with female luer IV line connectors. However, the health industry has not fixed this design flaw. Educating staffs on tubing misconnections and labeling tubing are temporary measures, not a solution.

Further, a review by the United States Pharmacopeia (USP) re-indicates that misconnection errors occurred with more than 300 cases reported to its database between 2000 and 2004. Thus, misconnections have caused serious and even fatal consequences, and while compliance with the current standard is voluntary, it is hoped that all hospitals and other health care facilities will eventually discard or discontinue the current adapter, which has an unsafe history of accidentally fitting into an IV line.

The objective of the present invention is to minimize this possibility of a potentially harmful connection to occur between enteral feeding sets with Central Intravenous Catheters, Central Venous pressure ports, Infusion ports, Balloon ports, Introducer ports, Peritoneal Dialysis Catheters, Distal Port for a Pulmonary Artery Catheter, IV Luer connectors or rigid female luer connectors, or intravenous (IV) catheter sets, and many other similar type fittings known within the industry. It is intended that this adapter may result in replacing the current industry standard, which may allow a new enteral feeding connector to be incompatible with Central Intravenous Catheters, Central Venous pressure ports, Infusion ports, Balloon ports, Introducer ports, Peritoneal Dialysis Catheters, Distal Port for a Pulmonary Artery Catheter, IV Luer connectors or rigid female luer connectors, or intravenous (IV) catheter sets, and many other similar type fittings known within the industry.

### SUMMARY OF THE INVENTION

One aspect of the present invention relates to a connector with a central opening adapted for passing fluid from a first end portion to a second end portion, with the first end portion being larger in diameter than the second end portion, one or more externally located ridges and one or more indentations positioned on the second end portion of the connector.

A further embodiment provides that the central opening is approximately 0.01 inches in diameter. One embodiment provides that the length of the connector is approximately 1.34 inches.

One embodiment provides that there is one or more externally ridges located on the connector. Yet, another embodiment provides that the ridge most proximate to the first end portion is larger in width than the successive ridges. Another embodiment provides that the larger ridge is approximately 0.433 inches in diameter. Furthermore, one embodiment provides that the distance between the ridges is approximately equal to, or less than, 0.20 inches.

Another embodiment provides that the connector further comprises a central shaft. A further embodiment provides that the central shaft is approximately 0.25 inches in diameter.

One embodiment of the present invention provides that the inlet located on the second end portion is greater than 0.166 inches wide. A further embodiment of the present invention provides that the inlet of the second end portion is equal to or less to 0.20 inches wide. Yet, another embodiment of the present invention provides that the notches located around the inlet are separated by approximately 0.045 inches.

One embodiment provides that the connector also comprises of one or more extensions located external to the first end portion, and fitted perpendicular to the central shaft. Yet, another embodiment provides that the extensions are approximately 0.03 inches in width.

Another aspect of the present invention provides a method for connecting tubes comprised of establishing a connection between a first end portion of a connector to a first tube, and a second end portion of the connector to a separate second tube, passing fluid through a central opening from the first end portion to the second end portion, and positioning an indention on the inlet of the central opening of the second end portion, wherein only a proper connection can be made between the second end portion of the connector and the second tube due to the size and notch on the inlet of the second end portion of the connector.

One embodiment of the invention provides that the central opening proximate to the first end portion of the connector contains a lip in order to secure connection of the tube within it.

### BRIEF DESCRIPTION OF THE DRAWINGS

FIG. 1A illustrates a side view of one embodiment of the present invention.

FIG. 1B illustrates a rotated side view of one embodiment of the present invention.

FIG. 2A illustrates a rear view of one embodiment of the present invention.

FIG. 2B illustrates a front view of one embodiment of the present invention.

FIG. 3 illustrates a cross-sectional view of one embodiment of the present invention.

### DETAILED DESCRIPTION OF THE PREFERRED EMBODIMENTS

Embodiments of the present invention will reflect the conscientious efforts of concerned health care professionals, device manufacturers, and government representatives, to develop a standard for those performance levels that could reasonably be achieved with this device.

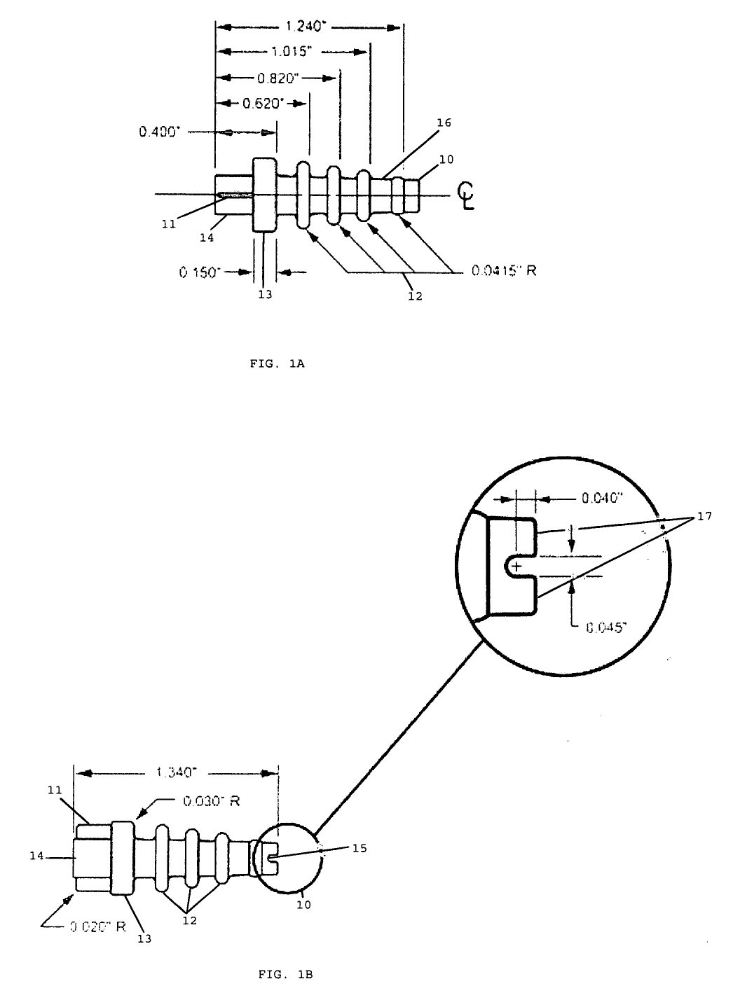

Referring to FIG. 1, a connector is provided in accordance with an embodiment of the present invention as illustrated. The connector has a first end portion 14 and a second end portion 10. The first end portion 14 is larger in diameter than the second end portion 10 due to fins 11, or peripherals, located on the external portion of the central shaft 16 of the connector. These fins 11 are utilized for screwing on and off the connector when it is attached the feeding tubes for use. The embodiment is shown to illustrate the present invention is equal to or less than 0.438 inches in diameter, 0.030 inches wide. The fins 11 may be larger or smaller in the length extending from the central shaft to offer more ease of use. As well, the fins 11 may be thicker or thinner in size. Also, the fins may also be slightly curved in order to provide a better grip by the user and greater comfort for the patient should the adapter be in contact with the patient's skin.

Next, the length of the connector may be approximately 1.34 inches. However, the length of the connector may be shortened or lengthened as needed. Located along the length of the connector are ridges 12 which are utilized to provide a seal in order to prevent enteral fluid from escaping the tube. Furthermore, the ridges 12 may be utilized to minimize the surface area, which makes it easier to remove the adapter at the end of a feeding cycle. The ridges 12 may be curved on the edges or straight. For purposes of the present invention, they are shown curved because this provides more comfort for the patient. The ridges 12 are external to the central shaft 16, and vary in diameter from the first end portion 14 to the second end portion 10, which may help identify which end portion is to be attached to the correct feeding tube. One embodiment of the present invention provides that there are five ridges, with the ridge 13 most proximate to the first end portion 14, being larger in width and diameter than those following it. The larger ridge 13 is approximately 0.150 inches wide and 0.438 inches in diameter. Each ridge maybe equidistantly separated by approximately 0.20 inches, however, this may vary dependent on the variable length of the overall connector.

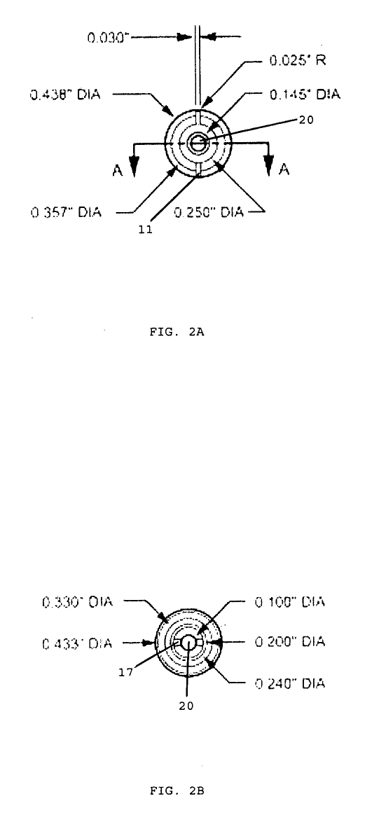

FIG. 2 provides a rotated view of FIG. 1A in order to further show the variation to the second end portion 10 of the connector. The second end portion 10 may be approximately equal to or less than 0.200 inches wide in diameter, not extending below 0.166 inches wide, as this may cause tube feed misconnections. In addition, the second end portion may include an indentation or gap 15 in order to ensure correct connection to the feeding tubes to the patient site, eliminating connections between the enteral feeding sets and the rigid female luer connectors, or intravenous (IV) catheter sets. The second end portion 10 may not be connected to any other tube, such as the enteral feeding set to a parenteral administration set, an indwelling catheter or port, an epidural catheter, a balloon inflation port, or tracheostomy tubes, because it will leak and/or not fit altogether, preventing this connection. This indention 15 may be approximately 0.040 inches deep, but may vary dependent on what is desired by industry standards in order to avoid tube misconnections. The indentation 15 may be approximately 0.045 inches wide, providing two separate finned portions 17 to be located around it. As well, there may be more than one indentation made, such that four separated sections are located around the central opening of the central shaft 16 of the connector.

FIGS. 2A and 2B provide rear and front views of the connectors, respectively. As shown, the indentation 17 located on the second end portion and the fins 11 located on the first end portion are located external to the central opening 20 of the connector. The central opening of the connector, which is utilized for liquid to flow through, may be approximately 0.100 inches in diameter. This may vary dependent on the length and size of the connector, but must remain greater than the size of the outer second end portion connection size of 0.166 inches in order to ensure safe connection to the feeding tube. Also, the sizes of the ridges in one embodiment of the present invention are provided. The ridges, as provided within an embodiment of the present invention, are approximately 0.438, 0.433, 0.330, 0.240, 0.200 in diameter, respectively. Each ridge, other than larger ridge 13, is approximately 0.0415 inches Radians wide. As previously mentioned, the number of ridges and sizes of the ridges may vary dependent on the overall length and variable size of the connector itself.

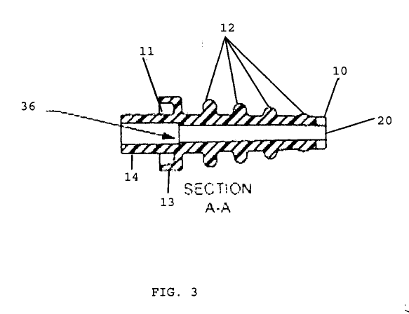

In addition, as shown in FIG. 3, the central opening 20, may have a lip 36, located more proximate to the first end portion 14 where the larger ridge 13 is located. The central shaft of the connector may be approximately 0.250 inches in diameter. The central shaft is approximately the same size from the first end portion to the second end portion, with the second end portion being slightly smaller in diameter at 0.200 inches.

The connector may be made of a plastic material or a similar type material as being utilized within industry standards. Further, the color of the connector, or adapter, may preferably be purple, which is currently of European standard. However, the color of the connector may vary dependent on color as desired by the local standards, or industry standards, at the time. In addition, a cap or top (not shown) may be utilized with the present invention in order to cover, or protect, the SafeFeed Adapter while not in use.

As previously described and shown within the detailed description of the embodiments of the present invention, the ultimate design goal of the Safe-Feed adapter is the prevention of any likelihood of unsafe connections between enteral feeding sets and devices other than enteral access devices. The SafeFeed adapter will minimize the possibility of potentially harmful connections occurring between enteral feeding sets and indwelling IV ports, catheters, peritoneal catheters, and tracheostomy tubes, etc. As well, the SafeFeed adapter may have other usages to reduce the likelihood of tube misconnections such as an adapter for blood pressure insufflator tubes.

While particular embodiments of the present invention have been disclosed, it is to be understood that various different modifications and combinations are possible and are contemplated within the true spirit and scope of the appended claims. There is no intention, therefore, of limitations to the exact abstract and disclosure herein presented.

What is claimed is:

1. A connector/adapter comprising:
a central opening adapted for passing fluid from a first end portion to a second end portion, the first end being larger diameter than the second end portion;
one or more external ridges, separated by and varying in diameter from the first end portion to the second end portion; and
one or more notches positioned on the inlet of the central opening of the second end portion.

2. The connector of claim 1 further comprising:
a central shaft on which the one or more external ridges are located.

3. The connector of claim 2 wherein the central shaft is approximately 0.25 inches in diameter.

4. The connector of claim 1 wherein the inlet of the second end portion is greater than 0.166 inches wide.

5. The connector of claim 1 wherein the inlet of the second end portion is equal to or less than 0.20 inches in diameter.

6. The connector of claim 1 wherein the one or more notches separations are approximately 0.045 inches wide.

7. The connector of claim 2 further comprising:
one or more extensions are located external to the first end portion for facilitating removal and coupling of the connector to tubing.

8. The connector of claim 7 wherein the extensions are positioned on the first end portion and fitted perpendicular to the central shaft.

9. The connector of claim 7 wherein the extensions are approximately 0.03 inches in width.

10. The connector of claim 1 wherein the length from the first end portion to the second end portion is approximately 1.34 inches.

11. The connector of claim 1 wherein the ridge proximate to the first end portion is larger in width than the successive ridges.

12. The connector of claim 11 wherein the ridge is approximately 0.433 inches in diameter.

13. The connector of claim 1 wherein the central opening is approximately 0.1 inches in diameter.

14. The connector of claim 1 wherein the distance between the one or more external ridges is equal to or less than 0.2 inches.

15. The connector of claim 1 wherein the central opening contains a lip proximate to the first end portion.

16. A method for connecting tubes comprising:
establishing a connection between a first end portion of a connector to a first tube and a second end portion of the connector to a separate second tube.
passing fluid through a central opening from the first end portion to the second end portion; and
positioning an indention on the inlet of the central opening of the second end portion;
wherein only a proper connection can be made between the second end portion of the connector and the second tube due to the size and notch on the inlet of the second end portion of the connector.

17. The method of claim 16 wherein the inlet on the second end portion of the connector is great than 0.166 inches.

18. The method of claim 16 wherein the central opening proximate to the first end portion of the connector contains a lip in order to secure the connection of the tube within it.
